(19) United States
(12) Patent Application Publication
(10) Pub. No.: US 2026/0224843 A1
(43) Pub. Date: Aug. 6, 2026

(54) LOCALIZED THERMAL PROTECTION OF MEDICAL DEVICES

(71) Applicant: Boston Scientific Scimed, Inc., Maple Grove, MN (US)

(72) Inventors: Narin Anderson, Shakopee, MN (US); Brian G. Williams, Prior Lake, MN (US); Keith Ervin Robertson, St. Michael, MN (US); Amit Kumar Rauniyar, Maple Grove, MN (US); Kirk Krona, Maple Grove, MN (US)

(73) Assignee: Boston Scientific Scimed, Inc., Maple Grove, MN (US)

(21) Appl. No.: 19/529,337

(22) Filed: Feb. 4, 2026

Related U.S. Application Data
(60) Provisional application No. 63/754,019, filed on Feb. 5, 2025.

(51) Int. Cl. A61M 25/00; A61F 2/00

(52) U.S. Cl. CPC A61M 25/002; A61F 2/0095; A61F 2250/0067; A61M 2205/0238; A61M 2209/06

(57) ABSTRACT

Devices for localized thermal protection of a medical device are disclosed. An example localized thermal protection device for use with a medical device may include a cylindrical body including a first layer and a second layer. The first layer may include a first substrate and a first phase change material disposed along the first substrate. The second layer may include a second substrate and a second phase change material disposed within the second substrate. The cylindrical body may be configured to be disposed about a discrete temperature-sensitive region of a medical device.

### CROSS-REFERENCE TO RELATED APPLICATIONS

This application claims the benefit of priority under 35 U.S.C. §119 of U.S. Provisional Application No. 63/754,019, filed February 5, 2025, the entire disclosure of which is hereby incorporated by reference.

### TECHNICAL FIELD

The present disclosure pertains to medical devices, and methods for manufacturing medical devices. More particularly, the present disclosure pertains to localized thermal protection devices of medical devices.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A localized thermal protection device for use with a medical device is disclosed. The localized thermal protection device comprises: a cylindrical body including a first layer and a second layer; wherein the first layer includes a first substrate and a first phase change material disposed along the first substrate; wherein the second layer includes a second substrate and a second phase change material disposed within the second substrate; and wherein the cylindrical body is configured to be disposed about a discrete temperature-sensitive region of a medical device.

Alternatively or additionally to any of the embodiments above, the first substrate, the second substrate, or both comprise a foam.

Alternatively or additionally to any of the embodiments above, the first substrate, the second substrate, or both the first substrate and the second substrate comprise high-density polyethylene.

Alternatively or additionally to any of the embodiments above, the first substrate and the second substrate are formed of the same material.

Alternatively or additionally to any of the embodiments above, the first substrate and the second substrate are formed of different materials.

Alternatively or additionally to any of the embodiments above, the first phase change material is configured to undergo a first phase change at a first temperature and the second phase change material is configured to undergo a second phase change at a second temperature different from the first temperature.

Alternatively or additionally to any of the embodiments above, the first phase change material is disposed along an outer surface of the first substrate.

Alternatively or additionally to any of the embodiments above, the first phase change material is embedded within the first substrate.

Alternatively or additionally to any of the embodiments above, the first substrate has a patterned region formed therein and wherein the first phase change material is disposed along the patterned region.

Alternatively or additionally to any of the embodiments above, the cylindrical body further comprises a third layer, the third layer includes a third substrate and a third phase change material disposed along the third substrate.

Alternatively or additionally to any of the embodiments above, the cylindrical body further comprises an aerogel layer, a vacuum insulated layer, or both.

Alternatively or additionally to any of the embodiments above, the discrete temperature-sensitive region of the medical device includes a drug coating.

Alternatively or additionally to any of the embodiments above, the discrete temperature-sensitive region of the medical device is disposed within a carrier tube and wherein the cylindrical body is disposed along an outer surface of the carrier tube.

A localized thermal protection device for use with a medical device is disclosed. The localized thermal protection device comprises: a cylindrical body including a first layer, a second layer disposed along the first layer, and a third layer disposed along the second layer; wherein the first layer includes a first foam substrate having a first phase change material embedded therein; wherein the second layer includes a second foam substrate having a second phase change material embedded therein; wherein the third layer includes a third foam substrate having a third phase change material embedded therein; wherein the cylindrical body is configured to be disposed about a discrete temperature-sensitive region of a medical device; wherein the discrete temperature-sensitive region of the medical device is disposed within a carrier tube; and wherein the cylindrical body is disposed along an outer surface of the carrier tube.

Alternatively or additionally to any of the embodiments above, the first foam substrate comprises high-density polyethylene.

Alternatively or additionally to any of the embodiments above, the first phase change material is configured to undergo a first phase change at a first temperature and the second phase change material is configured to undergo a second phase change at a second temperature different from the first temperature.

Alternatively or additionally to any of the embodiments above, the third phase change material is configured to undergo a third phase change at a third temperature different from the second temperature.

Alternatively or additionally to any of the embodiments above, the first phase change material is embedded within the first foam substrate.

Alternatively or additionally to any of the embodiments above, the first foam substrate has a patterned region formed therein and wherein the first phase change material is disposed along the patterned region.

A localized thermal protection device for use with a medical device is disclosed. The localized thermal protection device comprises: a cylindrical body including a first layer, a second layer disposed along the first layer, and a third layer disposed along the second layer; wherein an aerogel layer, a vacuum insulated layer, or both an aerogel layer and a vacuum insulated layer are disposed along the third layer; wherein the first layer includes a first foam substrate having a first phase change material embedded therein; wherein the second layer includes a second foam substrate having a second phase change material embedded therein; wherein the third layer includes a third foam substrate having a third phase change material embedded therein; wherein the first phase change material is configured to undergo a first phase change at a first temperature; wherein the second phase change material is configured to undergo a second phase change at a second temperature different from the first temperature; wherein the third phase change material is configured to undergo a third phase change at a third temperature different from the second temperature; and wherein the cylindrical body is configured to be disposed about a discrete temperature-sensitive region of a medical device.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example packaged medical device;

FIG. 2 illustrates the use of a localized thermal protection device with the medical device;

FIG. 3 is a cross-sectional view of the localized thermal protection device of FIG. 2;

FIG. 4 is a cross-sectional view of another example localized thermal protection device;

FIG. 5 is a cross-sectional view of another example localized thermal protection device; and

FIGS. 6-12 illustrate exemplary phase change material patterned regions formed with a substrate on the thermal protection device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

In a number of instances, a medical device may be temperature-sensitive and/or include a component that is temperature-sensitive. Such medical devices may need to be transported and/or stored in a manner that helps to maintain the desired temperature. Traditionally, protecting such medical devices has been accomplished using gross protection of an entire packaged medical device. For example, a packaged medical device (e.g., including a temperature-sensitive component) may be disposed within a cooler, insulated container, secondary box, and/or the like. This thermal protection of the entire packaged medical device may add size and/or weight (e.g., which may increase the storage and/or shipping cost), increase the complexity of the packaging, may be considered to be wasteful, and/or otherwise not considered to be environmentally friendly. Disclosed herein are localized thermal protection devices for medical devices that may help to improve the thermal protection of medical devices.

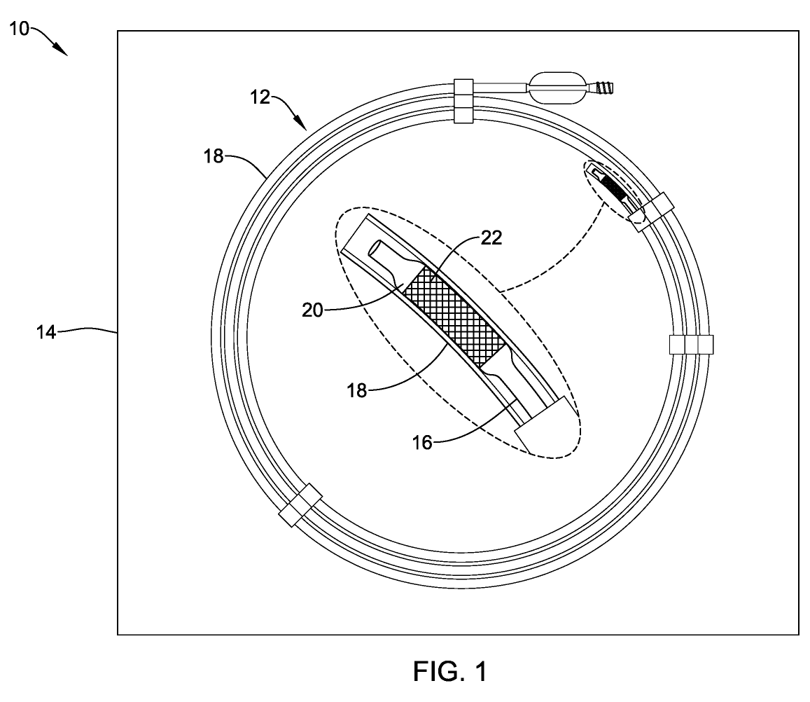

FIG. 1 illustrates an example packaged medical device 10. In this example, a medical device 12 is shown disposed within a package 14. The medical device 12 may include a catheter 16 disposed within a carrier tube 18. The catheter 16 may have a balloon 20 and/or a stent 22. In instances where the catheter 16 includes the balloon 20, a drug coating (not shown) may be disposed along the balloon 20. In instances where the catheter 16 includes the stent 22, a drug coating (not shown) may be disposed along the stent 22. The drug may be temperature-sensitive and/or otherwise be best stored in a manner that helps to reduce exposure to unwanted thermal conditions. A wide variety of additional medical devices may also benefit from thermal protection and may be used in conjunction with the localized thermal protection devices disclosed herein.

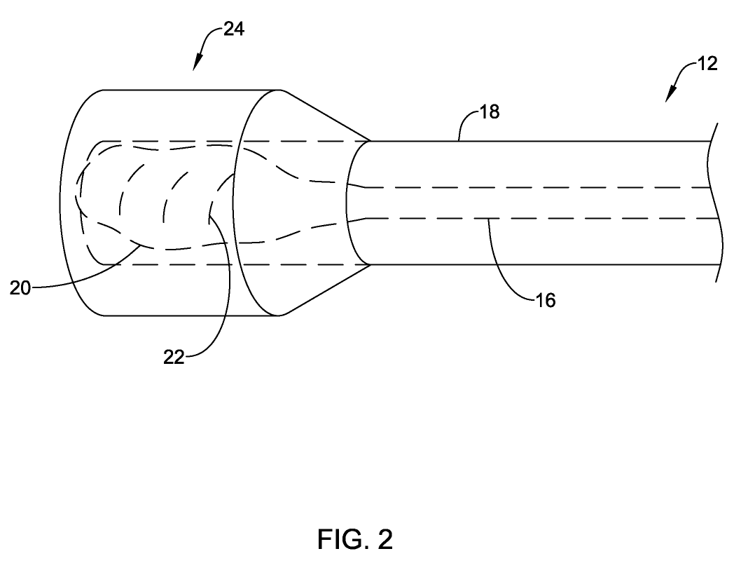

FIG. 2 illustrates the use of a localized thermal protection device 24 with the medical device 12. In this example, the localized thermal protection device 24 may take the form of a generally cylindrical body that, for example, may be disposed about the carrier tube 18 (e.g., a carrier tube 18 disposed about the catheter 16 and over the balloon 20 and/or the stent 22) in order to provide localized thermal protection. It can be appreciated that the localized thermal protection device 24 is only disposed along a discrete region of the medical device 12. For example, in this example, the localized thermal protection device 24 is shown disposed over the balloon 20 and/or the stent 22 (e.g., which may be drug coated and/or otherwise may include a thermally-sensitive region). By virtue of the localized thermal protection device 24 only being disposed over a discrete region of the medical device 12, it may be possible to minimize the impact of the localized thermal protection device 24 on overall size/weight the packaging of the medical device 12, may simplify packaging, may decrease the impact on the environment, etc.

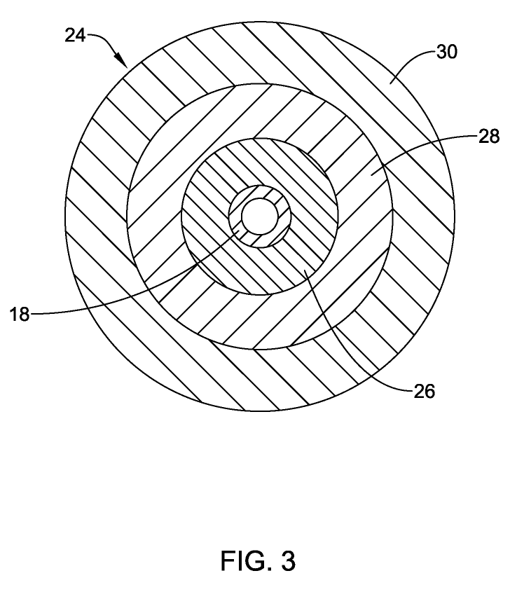

FIG. 3 is a cross-sectional view of the localized thermal protection device 24 that shows the form of the localized thermal protection device 24. In this example, the localized thermal protection device 24 is disposed about the carrier tube 18. However, this is merely schematic in nature and not intended to be limiting. As shown in FIG. 3, the localized thermal protection device 24 may include a plurality of layers. For example, the localized thermal protection device 24 may include a first layer 26 and a second layer 28 disposed about the first layer 26. In some instances, a third layer 30 may be disposed about the second layer 28. While three layers are shown in FIG. 3, this is not intended to be limiting. Localized thermal protection devices are contemplated that include one, two, three, four, five, six, or more layers. The layers may be uniform in thickness and construction or the layers may vary in thickness and/or construction. It can be appreciated that a variety of arrangements are contemplated for the layers utilized in the localized thermal protection device 24.

At least some of the layers 26, 28, 30 may include a base or substrate material and a phase change material. For the purposes of this disclosure, a phase change material may be understood to be a material that undergoes a phase change (e.g., solid to liquid) when exposed to particular thermal conditions and/or elevated temperature. The phase change material may allow the localized thermal protection device to be able to provide a signal that a particular thermal condition is present. For example, the phase change material may undergo a phase change at a particular temperature (e.g., the phase change material may change from a solid to a liquid). In instances where a visual que or indicator such as dye is combined with the phase change material, the layer containing the phase change material (and dye) may be an identifiable color. Changing the phase of the phase change material may cause the phase change material (and the dye) to migrate away from localized thermal protection device 24. When doing so, the localized thermal protection device 24 may no longer have the same color. The change in color may be observed by a user and can help to signify whether or not the medical device has been exposed to a particular temperature condition and/or is no longer considered to be suitable for use. It can be appreciated that when multiple layers are utilized where each layer includes a phase change material (and a dye), progressive color changes may occur that can help a user (e.g., a clinician) determine whether or not the medical device 12 has been exposed to particular (e.g. undesired) thermal conditions and/or is no longer considered to be suitable for use.

The layers 26, 28, 30 may be arranged with differing phase change materials and/or differing arrangements so that each layer may undergo a phase change when exposed to a particular temperature. For example, the first layer 26 (e.g., which may include a first base or substrate and a first phase change material) may be configured to undergo a phase change when exposed to a first temperature. The first temperature may vary and could be a temperature of about 10-50 °C, or about 15-30 °C, or about 23-24 °C. The second layer 28 (e.g., which may include a second base or substrate and a second phase change material) may be configured to undergo a phase change when exposed to a second temperature. The second temperature may be different from the first temperature. For example, the second temperature may be about 20-50 °C, or about 25-40 °C, or about 35 °C. The third layer 30 (e.g., which may include a third base or substrate and a third phase change material) may be configured to undergo a phase change when exposed to a third temperature. The third temperature may be different from the first and/or second temperature. For example, the third temperature may be about 25-70 °C, or about 35-60 °C, or about 45-50 °C. The variation in temperature responsiveness for the phase change materials may be accomplished by using different phase change materials, different ratios of phase change material to base/substrate, differing thickness of the layers, etc.

One example arrangement of the layers 26, 28, 30 may be arranged and function in the following manner. The outer layer 30 may include the highest melt point phase change material and may acts as first thermal barrier that can absorb the initial heat load. Upon exposure to elevated temperatures, the phase change material in the outer layer 30 may undergoes a gradual phase change and creates a temperature plateau at the specified/pre-determined melt point (e.g., about 25-70 °C, or about 35-60 °C, or about 45-50 °C, or about 50 °C). The middle layer 28 may include a phase change material with a melt point of about 20-50 °C, or about 25-40 °C, or about 35 °C. The middle layer 28 may remain solid while the outer layer 30 melts. This may help to provide a secondary thermal buffer. The middle layer only begins to undergo a phase change after the outer layer 30 saturates. This helps to maintain a temperature gradient. The inner layer 26 may have the lowest phase change material melt point (e.g., about 10-50 °C, or about 15-30 °C, or about 23-24 °C or about 24°C). The inner layer 26 may remain solid the longest and may help to maintain a stable temperature adjacent to the medical device disposed therein and benefits from thermal buffering of layers 28, 30. The phase change at the inner layer 26 occurs last.

The example arrangement described above may maximizes device protection by (a) creating steepest thermal gradient, (b) utilizing sequential phase changes, (c) providing staged heat absorption, and (d) maintaining the lowest temperature possible adjacent to the medical device. In summary, each layer 26, 28, 30 must fully melt before significant heat reaches the next adjacent layer, thus creating the longest possible protection duration. These effects could be further supplemented and reinforced by changing the ratio or incorporating differing layer thicknesses thus offering additional flexibility and granularity in our ability to program to a certain thermal protection profile.

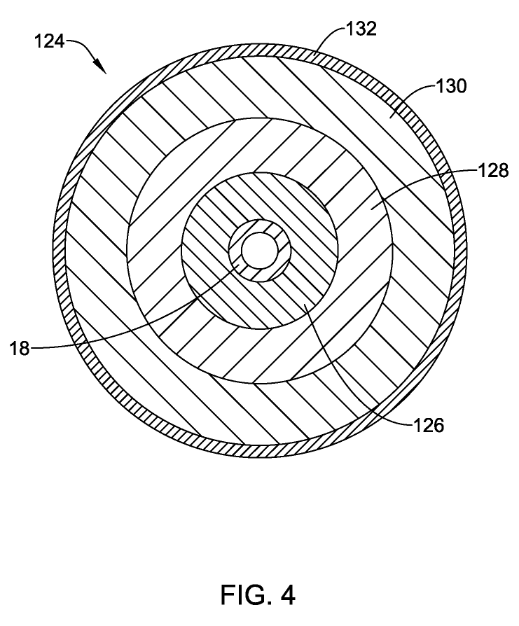

FIG. 4 is a cross-sectional view of another example localized thermal protection device 124 that may be similar in form and function to other localized thermal protection devices disclosed herein. In this example, the localized thermal protection device 124 may include a plurality of layers. For example, the localized thermal protection device 124 may include a first layer 126 and a second layer 128 disposed about the first layer 126. In some instances, a third layer 130 may be disposed about the second layer 128. In some instances, a fourth layer 132 may be disposed about the third layer 130. In this example, the fourth layer 132 may comprise an aerogel layer or a vacuum insulated layer.

In some instances, the fourth layer 132 may include or otherwise take the form of a wrap that can be disposed about one of the layers 126, 128, 130. The wrap may include a color changing material or dye, that changes color when exposed to a particular temperature. The color changing dye may be disposed within a stock paper or the like. In some instances, a top layer such as a clear protective tape, film, wax, and/or the like may be disposed about the wrap. When the temperature is breached, the melt point of the top layer is reached, thus causing the colorant in the indicator to migrate toward the surface thus allowing the user to visualize a color change.

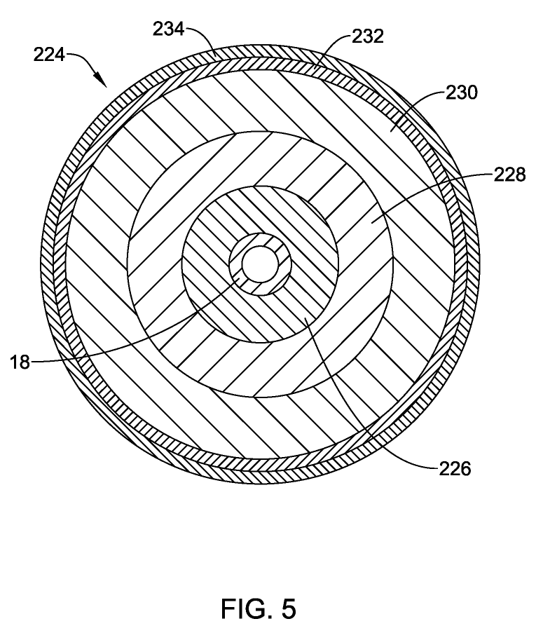

FIG. 5 is a cross-sectional view of another example localized thermal protection device 224 that may be similar in form and function to other localized thermal protection devices disclosed herein. In this example, the localized thermal protection device 224 may include a plurality of layers. For example, the localized thermal protection device 224 may include a first layer 226 and a second layer 228 disposed about the first layer 226. In some instances, a third layer 230 may be disposed about the second layer 228. In some instances, a fourth layer 232 may be disposed about the third layer 230. In some instances, a fifth layer 234 may be disposed about the fourth layer 232. In this example, the fourth layer 232 may comprise an aerogel layer or a vacuum insulated layer. Likewise, the fifth layer 234 may comprise an aerogel layer or a vacuum insulated layer.

The thermal protection devices disclosed herein, and/or the layers thereof, may be manufactured in a number of different manners. For simplicity, the manufacturing of the thermal protection devices is described using reference number 24. However, the discussion may be applied to any of the thermal protection devices disclosed herein (and/or the layers thereof). In some instances, the localized thermal protection device 24 may be manufactured from a suitable base or substrate material. For example, the base or substrate material may include a foam, high-density polyethylene, a high-density polyethylene foam, and/or other suitable materials. In some instances, each layer of the localized thermal protection device 24 may use the same material for the base/substrate material. In other instances, at least some of the layers of the localized thermal protection device 24 may differ from one another. The phase change material may be disposed along, embedded in, and/or otherwise coupled to the base material. In one specific example, not intended to be limiting, the base or substrate material may be 3-D printed and the phase change material may be applied as the base or substrate layer is formed. Other formation methodologies such as extrusion, casting, molding, etc. may be utilized.

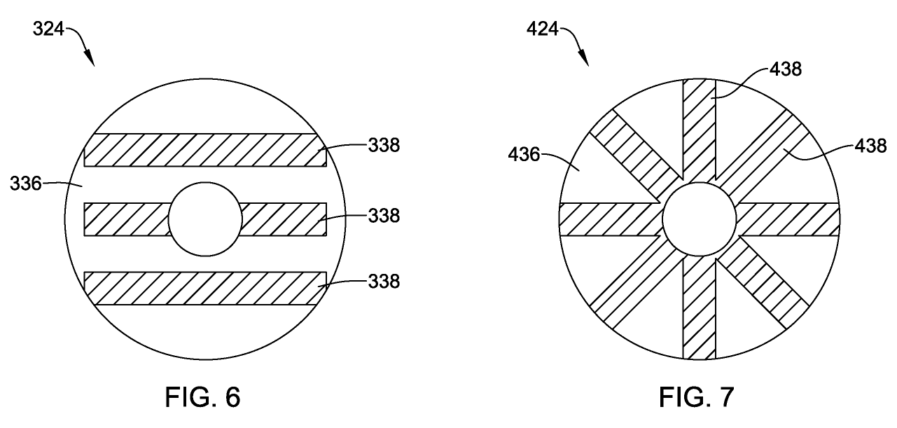

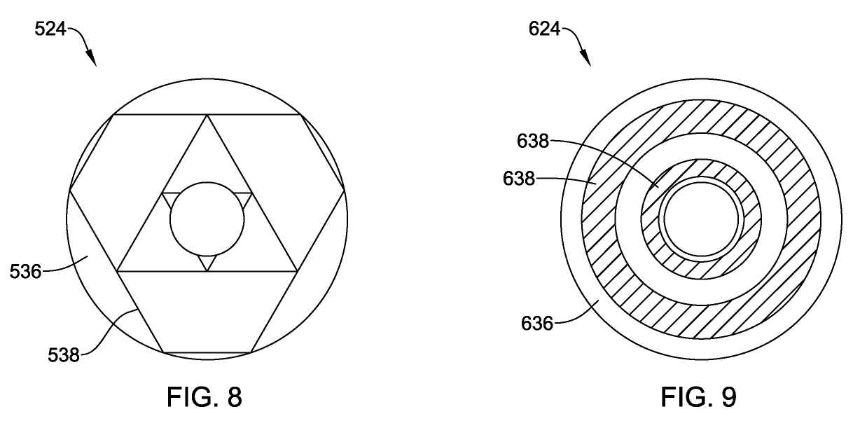

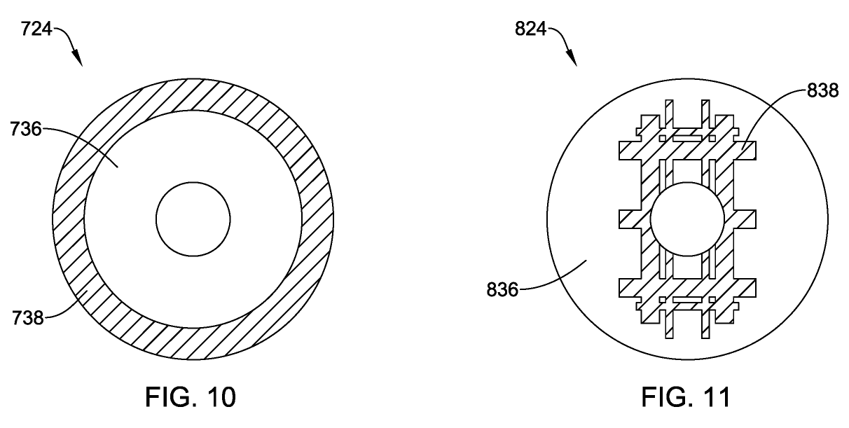

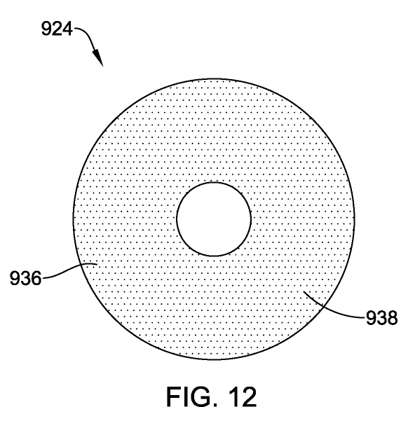

Because, for example, 3-D printing may be used to form the base or substrate material, it may be possible for the localized thermal protection devices and/or a layer thereof to be formed as a substrate with a patterned region formed therein. The patterned region may represent a location or distribution of the phase change material along the base or substrate material. FIGS. 6-12 illustrate at least some of the patterns contemplated. For example, FIG. 6 illustrates a thermal protection device 324 (and/or a layer of a thermal protection device 324) with a patterned region 338. The patterned region 338 may be characterized a plurality of longitudinal channels formed in the base or substrate material 336 where the phase change material is disposed. FIG. 7 illustrates a thermal protection device 424 (and/or a layer of a thermal protection device 424) with a patterned region 438, with the patterned region 438 characterized by a number of radial spikes formed in the base or substrate material 436 where the phase change material is disposed. FIG. 8 illustrates a thermal protection device 524 (and/or a layer of a thermal protection device 524) with a patterned region 538, with the patterned region 538 characterized by a honeycomb pattern formed in the base or substrate material 536 where the phase change material is disposed. FIG. 9 illustrates a thermal protection device 624 (and/or a layer of a thermal protection device 624) with a patterned region 638, with the patterned region 638 characterized by a plurality of concentric channels formed in the base or substrate material 636 where the phase change material is disposed. FIG. 10 illustrates a thermal protection device 724 (and/or a layer of a thermal protection device 724) with a patterned region 738, with the patterned region 738 characterized by spiral or helical channel formed in the base or substrate material 736 where the phase change material is disposed. FIG. 11 illustrates a thermal protection device 824 (and/or a layer of a thermal protection device 824) with a patterned region 838, with the patterned region 838 characterized by a random or fractal-like pattern formed in the base or substrate material 836 where the phase change material is disposed. FIG. 12 illustrates a thermal protection device 924 (and/or a layer of a thermal protection device 924) with a patterned region 938, with the patterned region 938 characterized by a gradient distribution pattern formed in the base or substrate material 936 where the phase change material is disposed. It can be appreciated that a wide variety of different patterns may be utilized without departing from the spirit of the disclosure. In at least some instances, the phase change material can be injected into a multicavity/patterned wall of a cylindrical form (e.g., the substrate material 336, 436, 536, 636, 736, 836, 936) and then can be sealed within the substrate material 336, 436, 536, 636, 736, 836, 936 using a suitable mechanism such as mechanical friction fit, laser welding with a cap, and/or the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A localized thermal protection device for use with a medical device, the localized thermal protection device comprising:
a cylindrical body including a first layer and a second layer;
wherein the first layer includes a first substrate and a first phase change material disposed along the first substrate;
wherein the second layer includes a second substrate and a second phase change material disposed within the second substrate; and
wherein the cylindrical body is configured to be disposed about a discrete temperature-sensitive region of a medical device.

2. The localized thermal protection device of claim 1, wherein the first substrate, the second substrate, or both comprise a foam.

3. The localized thermal protection device of claim 1, wherein the first substrate, the second substrate, or both the first substrate and the second substrate comprise high-density polyethylene.

4. The localized thermal protection device of claim 1, wherein the first substrate and the second substrate are formed of the same material.

5. The localized thermal protection device of claim 1, wherein the first substrate and the second substrate are formed of different materials.

6. The localized thermal protection device of claim 1, wherein the first phase change material is configured to undergo a first phase change at a first temperature and the second phase change material is configured to undergo a second phase change at a second temperature different from the first temperature.

7. The localized thermal protection device of claim 1, wherein the first phase change material is disposed along an outer surface of the first substrate.

8. The localized thermal protection device of claim 1, wherein the first phase change material is embedded within the first substrate.

9. The localized thermal protection device of claim 1, wherein the first substrate has a patterned region formed therein and wherein the first phase change material is disposed along the patterned region.

10. The localized thermal protection device of claim 1, wherein the cylindrical body further comprises a third layer, the third layer includes a third substrate and a third phase change material disposed along the third substrate.

11. The localized thermal protection device of claim 1, wherein the cylindrical body further comprises an aerogel layer, a vacuum insulated layer, or both.

12. The localized thermal protection device of claim 1, wherein the discrete temperature-sensitive region of the medical device includes a drug coating.

13. The localized thermal protection device of claim 1, wherein the discrete temperature-sensitive region of the medical device is disposed within a carrier tube and wherein the cylindrical body is disposed along an outer surface of the carrier tube.

14. A localized thermal protection device for use with a medical device, the localized thermal protection device comprising:
a cylindrical body including a first layer, a second layer disposed along the first layer, and a third layer disposed along the second layer;
wherein the first layer includes a first foam substrate having a first phase change material embedded therein;
wherein the second layer includes a second foam substrate having a second phase change material embedded therein;
wherein the third layer includes a third foam substrate having a third phase change material embedded therein;
wherein the cylindrical body is configured to be disposed about a discrete temperature-sensitive region of a medical device;
wherein the discrete temperature-sensitive region of the medical device is disposed within a carrier tube; and
wherein the cylindrical body is disposed along an outer surface of the carrier tube.

15. The localized thermal protection device of claim 14, wherein the first foam substrate comprises high-density polyethylene.

16. The localized thermal protection device of claim 14, wherein the first phase change material is configured to undergo a first phase change at a first temperature and the second phase change material is configured to undergo a second phase change at a second temperature different from the first temperature.

17. The localized thermal protection device of claim 16, wherein the third phase change material is configured to undergo a third phase change at a third temperature different from the second temperature.

18. The localized thermal protection device of claim 14, wherein the first phase change material is embedded within the first foam substrate.

19. The localized thermal protection device of claim 14, wherein the first foam substrate has a patterned region formed therein and wherein the first phase change material is disposed along the patterned region.

20. A localized thermal protection device for use with a medical device, the localized thermal protection device comprising:
a cylindrical body including a first layer, a second layer disposed along the first layer, and a third layer disposed along the second layer;
wherein an aerogel layer, a vacuum insulated layer, or both an aerogel layer and a vacuum insulated layer are disposed along the third layer;
wherein the first layer includes a first foam substrate having a first phase change material embedded therein;
wherein the second layer includes a second foam substrate having a second phase change material embedded therein;
wherein the third layer includes a third foam substrate having a third phase change material embedded therein;
wherein the first phase change material is configured to undergo a first phase change at a first temperature;
wherein the second phase change material is configured to undergo a second phase change at a second temperature different from the first temperature;
wherein the third phase change material is configured to undergo a third phase change at a third temperature different from the second temperature; and
wherein the cylindrical body is configured to be disposed about a discrete temperature-sensitive region of a medical device.
